(19) United States
(12) Patent Application Publication
(10) Pub. No.: US 2011/0052623 A1
(43) Pub. Date: Mar. 3, 2011

(54) DRIED MICROBIAL CELLS OR MICROORGANISM EXTRACT CONTAINING STABILIZED (SS)-S-ADENOSYL-L-METHIONINE AND METHOD FOR PRODUCTION OF THE DRIED MICROORGANISM CELL OR MICROORGANISM EXTRACT

(75) Inventors: Makoto Ueda, Takasago-shi (JP); Yui Kawashima, Takasago-shi (JP); Ryuuji Miki, Takasago-shi (JP); Takahiro Ueda, Osaka-shi (JP); Shiro Kitamura, Takasago-shi (JP); Hiroshi Kubo, Takasago-shi (JP)

(73) Assignee: KANEKA CORPORATION, OSAKA-SHI OSAKA (JP)

(21) Appl. No.: 12/809,474

(22) PCT Filed: Dec. 18, 2008
(86) PCT No.: PCT/JP2008/073051

§ 371 Date: Jun. 18, 2010

(30) Foreign Application Priority Data
Dec. 20, 2007 (EP) 2007-328220
Mar. 31, 2008 (JP) 2008-090254

(51) Int. Cl. A61K 36/06; A61P 25/28; A61P 25/24; A61P 1/16

(52) U.S. Cl. 424/195.16

(57) ABSTRACT

Dried microbial cells or microbial extract containing (SS)-SAM in which the progress in SAM chemical degradation or (SS)-SAM epimerization is significantly delayed can be obtained by drying microbial cells or microbial extract containing S-adenosyl-L-methionine (SAM), followed by maturing treatment. It is possible to stabilize SAM in a composition comprising dried microbial cells or microbial extract containing SAM (1% by weight or more) without the need of strict environment control.

### TECHNICAL FIELD

The present invention relates to dried microbial cells or microbial extract containing stabilized (SS)-S-adenosyl-L-methionine and a method for producing the same. Further, the present invention relates to a method for producing a composition comprising dried microbial cells or microbial extract containing S-adenosyl-L-methionine.

### BACKGROUND ART

S-adenosyl-L-methionine (hereinafter abbreviated as SAM) is a substance that is widely present in biological tissues and involved in many biological reactions as an enzyme-activating factor or a methyl-group donor for synthesis/metabolism of nucleic acids, neurotransmitters, phospholipids, hormones, proteins, and the like. In addition, SAM is known to be effective for alcoholic hepatitis, other liver disorders, depression, osteoarthropathy, and senile dementia (Non-Patent Documents 1 and 2 and Patent Documents 1 and 2).

In general, SAM is a mixture comprising two types of diastereoisomers, which are (SS)-S-adenosyl-L-methionine (hereinafter abbreviated as (SS)-SAM) and (RS)-S-adenosyl-L-methionine (hereinafter abbreviated as (RS)-SAM). However, it is known that, of the two types of diastereoisomers, only (SS)-SAM has enzymatic methyl transfer reaction activity, and that inert (RS)-SAM is produced via nonenzymatic epimerization of (SS)-SAM (Non-Patent Documents 3 to 5).

SAM is known as a highly chemically unstable substance that is rapidly degraded even at ordinary temperatures. Due to this characteristic, the total content of SAM and the proportion of (SS)-SAM, which is activated form, among the total SAM content (diastereomer ratio) decrease over time during preservation or distribution. In addition, when SAM is subjected to processing treatment such as formulation preparation in an environment of ordinary temperature and humidity, SAM stability significantly decreases during long-term preservation following processing, even if SAM degradation can be prevented during processing. This is a significant obstacle in terms of quality assurance or production of SAM when it is used as pharmaceutical product or supplement.

In view of the above reasons, methods for stabilizing SAM by forming SAM into a salt with the use of an acid such as an inorganic acid (e.g., phosphoric acid, polyphosphoric acid, metaphosphoric acid, hydrochloric acid, or sulfuric acid), an organic sulfonic acid derivative (e.g., p-toluenesulfonic acid), strong acid or an organic acid (e.g., acetic acid, lactic acid, citric acid, or succinic acid) have been suggested. Furthermore, there are known methods for preventing SAM degradation by further adding an inorganic salt (e.g., magnesium sulfate or calcium chloride) or an organic compound (e.g., ascorbic acid, maltose, cyclodextrin, or acylated taurine derivative) to an SAM salt described above (Patent Documents 3 to 14 and Non-Patent Documents 6 to 9).

Also, there are known example methods comprising adding a compound such as citric acid, succinic acid, kojic acid, EDTA, a phosphoric acid compound, or trehalose to microbial cells or microbial extract containing SAM for similar purposes (Patent Documents 15 and 16 and Non-Patent Document 10)

However, in the past reports described above, the degree of SAM stabilization is evaluated simply based on the total content of SAM containing (RS)-SAMwhich is non-activated form but not based on the content of (SS)-SAM (activated form) in terms of purity, which should be calculated in view of the diastereomer ratio.

Moreover, the stability of SAM produced in a salt form by the above methods is not satisfactory for production of SAM as a pharmaceutical product or supplement. In the above cases, production must be carried out in a strictly controlled environment.

In addition, there are SAM products practically available in, for example, Western countries as pharmaceutical products and functional foods, which are combined salts of SAM, p-toluenesulfonic acid, and sulfuric acid and salts of SAM and 1,4-butanedisulfonic acid. However, in Japan, the additives used in such products are not accepted as, for example, food additives. Therefore, a method for further stabilizing SAM for use in a food or supplement has been required.

Establishment of stabilization technology that will overcome all the above problems is still a very difficult technical object. Since it is impossible to impart SAM stability in terms of practical durability, it is not always possible to undertake sufficient discussion regarding properties other than stability (e.g., absorbability and efficacy of formulations containing SAM and the usability of SAM in various fields).

Further, a chemical synthesis method and a method comprising culturing a microorganism (e.g., a yeast of the genus Saccharomyces) followed by extraction and purification (Patent Documents 17 and 18) have been known for the industrial production of SAM. The latter is a more appropriate method of producing SAM with a high diastereomer ratio of (SS)-SAM (activated form).

However, due to the instability of (SS)-SAM generated during culture (degradation and epimerization), it is difficult to preserve microbial cells or microbial extract containing SAM. Therefore, for further processing, it is necessary to obtain microbial cells and then immediately carry out the subsequent steps for formulation preparation.

### Patent Document 1: JP Patent Publication (Kokai) No. 2005-261361 A

### Patent Document 2: US 2003-144244

### Patent Document 3: EP 141914

### Patent Document 4: EP 162323

Patent Document 5: U.S. Pat. No. 4,028,183

### Patent Document 6: JP Patent Publication (Kokai) No. 51-125717 A (1976)

### Patent Document 7: JP Patent Publication (Kokai) No. 56-99796 A (1981)

### Patent Document 8: JP Patent Publication (Kokai) No. 56-92899 A (1981)

### Patent Document 9: JP Patent Publication (Kokai) No. 57-134499 A (1982)

### Patent Document 10: JP Patent Publication (Kokai) No. 57-156500 A (1982)

### Patent Document 11: JP Patent Publication (Kokai) No. 60-181095 A (1985)

### Patent Document 12: JP Patent Publication (Kokai) No. 59-108799 A (1984)

### Patent Document 13: JP Patent Publication (Kokai) No. 61-189293 A (1986)

### Patent Document 14: EP 395656

### Patent Document 15: JP Patent Publication (Kokai) No. 2005-229812

### Patent Document 16: JP Patent Publication (Kokai) No. 2007-197346

### Patent Document 17: JP Patent Publication (Kokai) No. 58-146291 A (1983)

### Patent Document 18: JP Patent Publication (Kokai) No. 58-138393 A (1983)

### Non-Patent Document 1: Hepatology, 1990, 111, 65

Non-Patent Document 2: J. Fam. Pract., 2002, 51, 425
Non-Patent Document 3: J. Am. Chem. Soc., 1959, 81, 3975
Non-Patent Document 4: J. Am. Chem. Soc., 1977, 99, 7292

### Non-Patent Document 5: Biochemistry, 1983, 22, 2828

Non-Patent Document 6: J. Biol. Chem., 1957, 229, 1037

### Non-Patent Document 7: J. Bacteriol., 1975, 121, 267

Non-Patent Document 8: Agric. Biol. Chem., 1984, 48, 2293

### Non-Patent Document 9: Research Disclosure, 1991, December, 927

### Non-Patent Document 10: Biochemica et Biophysica Acta, 2002, 1573, 105

### DISCLOSURE OF THE INVENTION

### Problem to be Solved by the Invention

Examination conducted by the present inventors showed that the stability of (SS)-SAM in its activated form (in terms of purity) was far from sufficient, although there are many methods that have been reported to have stabilizing effects. Therefore, the present inventors believe that the insufficient stability of (SS)-SAM in terms of purity is the reason why most of the variety of past attempts to stabilize SAM have not resulted in practical use of SAM. It is an object of the present invention to provide a method for stabilizing SAM that can be safely used for foods and supplements, and particularly, a method for stabilizing (SS)-SAM (activated form). It is another object of the present invention to provide dried microbial cells or microbial extract containing stabilized (SS)-SAM that can be preferably subjected to further processing and thus is excellent in terms of industrial-scale productivity and economic efficacy and to provide a method for stabilizing the same.

Further, it is another object of the present invention to provide a method for processing SAM that is excellent in terms of industrial-scale productivity and economic efficacy or a composition containing SAM by preventing degradation of SAM.

### Means for Solving Problem

In view of the above circumstances, the present inventors have thoroughly studied the essence of stabilization of (SS)-SAM contained in microbial cells or microbial extract in order to use microbial cells or microbial extract containing (SS)-SAM for foods, supplements, or the like at low cost. In addition, they have intensively studied a method for stabilizing SAM.

As a result, they have found that (SS)-SAM degradation and epimerization can be significantly prevented or discontinued by carrying out maturing treatment of microbial cells or microbial extract containing (SS)-SAM, and thus (SS)-SAM can be stabilized without the use of excessive amounts of additives.

In addition, they have found that microbial cells capable of exhibiting high absorbability in animal experiments can be obtained by carrying out a specific treatment (drying) and without carrying out a complicated and expensive treatment.

Further, they have found that the (SS)-SAM stability can be further improved by adding a metal salt and/or an ammonium salt to microbial cells or microbial extract containing (SS)-SAM.

Moreover, they newly found that reduction of SAM stability can be prevented not only during processing but also during long-term preservation after processing with the use of a composition comprising dried microbial cells or microbial extract containing SAM at 1% by weight or more.

Specifically, the present invention encompasses a method for producing dried microbial cells containing (SS)-SAM, comprising culturing a microorganism capable of producing (SS)-SAM, followed by drying microbial cells, and maturing the cells.

In addition, the present invention encompasses a method for producing microbial extract containing (SS)-SAM, comprising culturing a microorganism capable of producing (SS)-SAM, followed by disrupting microbial cells, drying a microbial extract from which solid matter has been removed according to need, and maturing the resultant.

Further, the present invention encompasses dried microbial cells or microbial extract containing stabilized (SS)-SAM produced by the above method.

Further, the present invention encompasses a composition comprising the dried microbial cells or microbial extract containing (SS)-SAM, which is orally administered.

Furthermore, the present invention encompasses a method for preserving dried microbial cells and/or microbial extract containing (SS)-SAM, comprising wrapping/packaging dried microbial cells or microbial extract containing (SS)-SAM with a glass, plastic, and/or metal material.

Furthermore, the present invention encompasses a method for increasing the (SS)-SAM concentration in plasma by orally administering dried microbial cells and/or microbial extract containing (SS)-SAM.

Furthermore, the present invention encompasses a method for preventing (SS)-SAM degradation and/or epimerization, comprising adding a metal salt and/or an ammonium salt to microbial cells or microbial extract containing (SS)-SAM so as to allow the salt to coexist therewith.

Furthermore, the present invention encompasses a method for producing an SAM-containing formulation containing a composition comprising dried microbial cells or microbial extract containing SAM at 1% by weight or more.

In addition, the present invention encompasses the above production method comprising carrying out formulation preparation at a relative humidity of 70% RH or less.

Also, the present invention encompasses the above production method comprising carrying out formulation preparation at 50° C. or less.

Further, the present invention encompasses the above production method, wherein formulation preparation is carried out by exposing a composition containing dried microbial cells and microbial extract containing SAM to a humidity of 20% RH or more and a temperature 20° C. or more for 96 hours or less.

### EFFECTS OF THE INVENTION

The present invention provides dried microbial cells or microbial extract containing stabilized (SS)-SAM and a method for producing the same. Such dried microbial cells or microbial extract containing stabilized (SS)-SAM can be directly used or processed into the form of a different formulation (e.g., tablets, chewable tablets, or capsules) and used for foods and supplements (e.g., health foods and functional foods) in a preferable manner.

In addition, in the present invention, a composition containing dried microbial cells or microbial extract containing SAM is processed in order to prevent degradation of highly unstable SAM not only during processing but also after processing. Thus, it has become possible to provide a method for producing or processing a composition comprising dried microbial cells or microbial extract containing SAM, comprising preventing SAM degradation in a simple way without strictly controlling a surrounding environment.

### BRIEF DESCRIPTION OF THE DRAWINGS

FIG. 1 shows changes in the SAM concentration in plasma after oral administration of dried yeast cells obtained in Example 4.

FIG. 2 shows changes in the SAM concentration in plasma after oral administration of dried yeast cells obtained in Example 5.

### BEST MODE FOR CARRYING OUT THE INVENTION

The present invention is hereafter described in greater detail with reference to the following embodiments, although the technical scope of the present invention is not limited thereto.

### (Microorganism)

Any microorganism can be used in the present invention as long as it can produce SAM. However, a microorganism containing SAM at 1% by weight or more in terms of dry weight is preferable. In addition, a microorganism containing SAM at 5% by weight or more in terms of dry weight is more preferable. A microorganism containing SAM at 10% by weight or more in terms of dry weight is further preferable. Since such microorganism is used for foods and supplements, it is preferably a microorganism that has been used as an edible product.

Microorganisms used in the present invention are not particularly limited. However, examples thereof include microorganisms belonging to the genera Saccharomyces, Candida, Pichia, Mucor, Rhizopus, Brevibacterium, Corynebacterium, Escherichia, and Streptomyces. Microorganisms belonging to the genus Saccharomyces are preferable. Of these microorganisms, those that have been used as edible products are preferable. Specific examples thereof include microorganisms such as sake yeast, bakers' yeast, beer yeast, and wine yeast. A more preferable example is the Saccharomyces cerevisiae K-6 strain (sake yeast kyokai no. 6). The Saccharomyces cerevisiae K-6 strain can be obtained as the NBRC2346 strain from the National Institute of Technology and Evaluation. In addition, microorganisms used in the present invention may be wild strains of the above microorganisms or mutant strains which are mutated or improved wild strains. Such mutant strains can be obtained by methods known by those skilled in the art which involve UV irradiation or treatment with the use of agents such as N-methyl-N′-nitro-N-nitrosoguanidine (NTG) and ethyl methanesulphonate (EMS). Further, it is also possible to use transformed microorganisms produced by a gene recombinant method or the like in a manner such that such microorganisms can produce SAM at high productivity.

### (Culture)

Microbial cells containing SAM used in the present invention can be obtained by culturing microorganisms described above by known methods. For example, in accordance with the method of Schlenk et al. described in Journal of Biological Chemistry, 1987, 29, 1037, such microorganisms can be cultured in liquid media containing methionine, carbon sources, nitrogen sources, and inorganic salts. Addition of organic micronutrients (e.g., vitamins and amino acids) to such media often results in preferable outcomes. Examples of carbon sources that can be appropriately used include: carbohydrates such as glucose and sucrose; organic acids such as acetic acid; and alcohols such as ethanol. Examples of nitrogen sources that can be used include ammonium salts, ammonia water, ammonia gas, urea, yeast extract, peptone, and corn steep liquor. Examples of inorganic salts that can be used include phosphate, magnesium salts, potassium salts, sodium salts, calcium salts, iron salts, sulfate, and chloride salts.

In addition, stabilizing agents described below, which have effects of preventing SAM chemical degradation and/or (SS)-SAM epimerization, may be added during culture.

Culture can be carried out under either aerobic conditions or anaerobic conditions. However, for efficient proliferation of microbial cells, aerobic conditions are preferable.

The culture temperature may fall within the range in which microorganisms can proliferate. However, the culture temperature is preferably 15° C. to 40° C. and more preferably 25° C. to 35° C. In addition, the pH during culture may fall within the range in which microorganisms can proliferate. However, culture is carried out preferably at pH 3 to 8 and more preferably at pH 5 to 7. Further, either a batch culture method or a continuous culture method may be used.

For the control of the above pH, either acid or alkali can be added. Examples of acid include: inorganic acids such as hydrochloric acid, sulfuric acid, and phosphoric acid; and organic acids such as acetic acid and citric acid. Examples of alkali include: carbonates such as potassium carbonate and sodium carbonate; alkali metal hydroxides such as sodium hydroxide, potassium hydroxide, and lithium hydroxide; alkaline-earth metal hydroxides such as magnesium hydroxide and calcium hydroxide; and ammonia. The above examples may be used alone or in combination of two or more in a mixture form.

For instance, in a case in which SAM is produced with the use of the Saccharomyces cerevisiae K-6 strain, a medium containing sucrose, yeast extract, L-methionine, urea, glycine, potassium dihydrogen phosphate, magnesium sulfate heptahydrate, biotin, calcium chloride dihydrate, and trace metal salts is inoculated with the strain, followed by aerobic culture at 30° C. for 4 days during feeding of a carbon source such as sucrose and/or ethanol. Thus, microbial cells containing SAM can be obtained.

### (Concentration/Washing/Disruption)

Microbial cells obtained by culture may be concentrated, separated or washed from the medium components, according to need by a method involving centrifugation, filtration, or the like.

A microbial extract containing SAM can be obtained by disrupting microbial cells cultured by the above method and, if necessary, removing undissolved matter by a means of centrifugation, filtration, or the like. A method for disrupting microbial cells is not particularly limited. However, examples of such method include: a method for disrupting by high-pressure disruption via high-pressure dispersion treatment or the like; a method for disrupting by mechanical disruption by means of a bead mill or the like; a method for disrupting by adding an organic solvent such as ethanol or ethyl acetate; a method for disrupting by adding acid or alkali; a method for disrupting by adding a surfactant; a method for disrupting by freeze-thawing; a method for disrupting by heat treatment; a method for disrupting by using a protease, a cell wall lytic enzyme, or the like; and a method for disrupting by inducing autolysis with the use of an enzyme contained in yeast. Preferred examples of such method include: a method for disrupting by high-pressure disruption via high-pressure dispersion treatment; a method for disrupting by adding an organic solvent; a method for disrupting by adding acid or alkali; and a method for disrupting by heat treatment. Needless to say, two or more methods selected from among the above methods can be used in combination.

### (Drying/pH)

Next, drying of microbial cells or microbial extract obtained by the above method is carried out. A drying method is not particularly limited. However, examples thereof include spray drying, lyophilization, reduced-pressure drying, and through-flow drying. In addition, two or more methods selected from among the above methods can be used in combination.

For spray drying, a spray drying method can be used.

The drying temperature is not particularly limited in the case of reduced-pressure drying or through-flow drying. However, in view of SAM stability, the temperature is preferably 60° C. or less. The lower limit of temperature is not particularly limited.

The pH of a microbial cell suspension or a microbial cell extract solution of microbial cells or microbial extract prior to drying is preferably 7 or less, more preferably 6 or less, and particularly preferably 5 or less.

For the control of the pH, either acid or alkali can be added. Examples of acid include, but are not particularly limited to: inorganic acids such as hydrochloric acid, sulfuric acid, and phosphoric acid; and organic acids such as acetic acid and citric acid. Examples of alkali include, but are not particularly limited to: carbonates such as potassium carbonate and sodium carbonate; alkali metal hydroxides such as sodium hydroxide, potassium hydroxide, and lithium hydroxide; alkaline-earth metal hydroxides such as magnesium hydroxide and calcium hydroxide; and ammonia. The above examples may be used alone or in combination of two or more in a mixture form.

In order to obtain maximum effects of the present invention, it is preferable to avoid, as much as possible, exposure to high temperature during drying. Reduced-pressure drying or through-flow drying requires a relatively long period of time (e.g., 6 hours or more). Therefore, drying is carried out at preferably at 30° C. or less and more preferably 20° C. or less. In view of productivity and product quality, preferable drying methods are a lyophilization method that can be carried out at low temperatures and a spray drying method wherein exposure to a relatively high-temperature environment is carried out for a very short period of time. A lyophilization method is more preferable.

Further, in order to reduce the moisture content in microbial cells and/or microbial extract, another drying (e.g. final drying) can be carried out by, for example, a depressurization, aeration, or heating operation after the above drying operation.

In order to obtain maximum maturing effects and maximum SAM stabilizing effects of the present invention, the moisture content in microbial cells and/or microbial extract upon the termination of drying is preferably at a minimum level. In general, the moisture content is 10% or less, preferably 5% or less, more preferably 3% or less, further preferably 2% or less, and particularly preferably 1% or less.

### (Maturing)

The dried microbial cells and/or microbial extract containing stabilized (SS)-SAM of the present invention can be readily produced by, for example, maturing dried microbial cells and/or microbial extract containing (SS)-SAM by the above method.

The term “maturing” indicates placement of dried microbial cells or microbial extract containing (SS)-SAM in a specific environment isolated from the external environment. Such specific environment refers to an environment in which a solid and/or gas substance can exclusively exist under specific temperature and pressure conditions (that is to say, substantially no liquefiable substance can exist). Such specific environment can be created in a manner described below.

1. The inside of an apparatus is depressurized and then the internal environment of the apparatus is isolated from the external environment while the depressurized state is maintained.
2. A dried gas such as nitrogen gas (preferably dried inert gas) is introduced into an apparatus, the air contained in the apparatus is substituted with the dried gas, and then the internal environment of the apparatus is isolated from the external environment.
3. A desiccant such as silica gel is introduced into an apparatus and then the internal environment of the apparatus is isolated from the external environment.

Needless to say, a plurality of the above operations can be used in combination.

In addition, isolation from the external environment herein defined means the maintenance of the above specific environment that has been created by, for example, controlling a valve of an apparatus such as a reaction can, a dryer, or a desiccator at least during the period required for maturing. It is also possible to create an environment completely isolated from the external environment via wrapping/packaging described below for complete sealing.

The temperature during maturing is not particularly limited. However, maturing can be carried out at generally 60° C. or less, preferably 50° C. or less, and more preferably 40° C. or less. In addition, maturing can be carried out at generally −80° C. or more, −60° C. or more, −40° C. or more, or −20° C. or more, preferably 0° C. or more, and more preferably 10° C. or more.

The time required for maturing is not particularly limited. However, it is generally 1 hour or more, preferably 2 hours or more, more preferably 5 hours or more, and particularly preferably 10 hours or more. The upper limit of the time is not particularly limited. However, if the time required for maturing is long, productivity decreases. Therefore, it is generally 30 days or less, preferably 15 days or less, more preferably 7 days or less, and further preferably 5 days or less.

The relative humidity during maturing is preferably at a minimum level. It is generally 30% RH or less, preferably 20% RH or less, more preferably 15% RH or less, and particularly preferably 10% RH or less.

In order to obtain maximum effects of the present invention, it is preferable to carry out the maturing operation shortly after the end of the previous step of drying. It is preferable to carry out maturing within generally 24 hours or less, preferably 12 hours or less, and more preferably 6 hours after the end of drying. Needless to say, it is also possible to carry out drying and maturing in a continuous/combined operation. Specifically, an apparatus used for drying is continuously used for maturing. In a case in which the internal environment of the apparatus is isolated from the external environment during drying, isolation is maintained. Alternatively, in a case in which the internal environment thereof is not isolated from the external environment during drying, the internal environment is isolated after drying such that maturing can be carried out continuously after drying.

Note that, in view of minimization of SAM degradation or epimerization, it is necessary for microbial cells or microbial extract containing SAM not to be exposed to high temperatures for many hours before being dried to the above moisture content level. For instance, the time required for exposure to a temperature exceeding 20° C. is generally 24 hours or less, preferably 12 hours or less, and more preferably 6 hours or less. In a particularly preferred embodiment, lyophilization of microbial cells or microbial extract containing SAM is carried out by a general method, followed by maturing. During maturing after lyophilization, it is possible to continuously maintain the temperature and the degree of depressurization for drying. Also, it is possible to maintain the depressurized environment and discontinue the temperature control (i.e., gradual increase of the temperature to room temperature) or to increase the pressure inside an apparatus to ordinary pressure with the use of a dried inert gas and maintain the temperature at an appropriate level.

In the above case, the (SS)-SAM content in dried microbial cells or microbial extract upon the end of the maturing operation is preferably 3% by weight or more, more preferably 5% by weight or more, further preferably 10% by weight or more, and particularly preferably 15% by weight or more in terms of dry weight.

(SS)-SAM can be stabilized without the use of an additive in the case of dried microbial cells or microbial extract containing SAM obtained by the method of the present invention.

### (Absorbability)

Dried microbial cells containing SAM obtained by the present invention show excellent SAM bioabsorbability comparable to or exceeding p-toluenesulfonic acid/sulfate bioabsorbability. In the case of oral administration of dried microbial cells or microbial extract containing SAM of the present invention at a dose of 100 mg/kg body weight in terms of SAM purity, the maximum SAM concentration in plasma can be 0.1 μg/ml or more, preferably 0.2 μg/ml or more, more preferably 0.3 μg/ml or more, and further preferably 0.5 μg/ml or more. In addition, the area under curve (AUC) of the SAM concentration is 0.7 (μg/ml)×h or more, preferably 1.0 (μg/ml)×h or more, more preferably 2.0 (μg/ml)×h or more, and further preferably 3.0 (μg/ml)×h or more.

Therefore, it can be expected that administration of the dried microbial cells or microbial extract of the present invention be highly effective for the improvement of liver functions and diseases/or pathological conditions such as depression, osteoarthropathy, and senile dementia for which SAM is supposed to be effective.

### (Stabilizing Agents)

In addition, in order to further improve the stability of SAM contained in the dried microbial cells or microbial extract of the present invention, it is possible to add a stabilizing agent having effects of preventing SAM chemical degradation and/or (SS)-SAM epimerization to the microbial cells or microbial extract during culture of a microorganism, before drying, or after drying.

The above stabilizing agent is not particularly limited as long as it has a feature of preventing of SAM chemical degradation and/or (SS)-SAM epimerization. However, it is preferably made from food material in view of safety for the use of the microbial cells or microbial extract containing SAM obtained by the present invention as a food, a health food, or a functional food or in a mixture comprising a food, a health food, or a functional food.

Examples of stabilizing agents include: inorganic acids (e.g., hydrochloric acid, sulfuric acid, sulfurous acid, phosphoric acid, pyrophosphoric acid, polyphosphoric acid, and metaphosphoric acid) and salts thereof; organic acids (e.g., acetic acid, citric acid, malic acid, succinic acid, tartaric acid, gluconic acid, fumaric acid, L-ascorbic acid, nicotinic acid, pantothenic acid, phytic acid, tosylic acid, and p-toluenesulfonic acid) and salts thereof; sugars (e.g., glucose, sucrose, fructose, galactose, trehalose, D-cellobiose, mannitol, inositol, inulin, fructooligosaccharide, isomaltooligosaccharide, cellulose, N-acetylglucosamine, sorbose, glycogen, dulcitol, lactitol, galactitol, gluconolactone, alginic acid, carrageenan, dextrin, erythritol, sorbitol, and xylitol) and derivatives thereof; amino acids or peptides (e.g., glycine, L-alanine, L-valine, L-leucine, D-leucine, L-threonine, L-methionine, DL-methionine, L-glutamine, L-glutamic acid, L-aspartic acid, L-ornithine, glutathione, and DL-α-amino-n-butyric acid) and salts thereof.

Examples of inorganic acid salts include metal salts and ammonium salts such as ammonium chloride, ferric chloride, magnesium chloride, calcium chloride, aluminium chloride, aluminium copper sulfate, ferrous sulfate, magnesium sulfate, calcium sulfate, manganese sulfate, nickel sulfate, zinc sulfate, copper sulfate, ammonium sulfate, sodium sulfate, aluminium sulfate, aluminium potassium sulfate, aluminium ammonium sulfate, ammonium carbonate, sodium carbonate, sodium phosphate, magnesium phosphate, calcium phosphate, ammonium phosphate, sodium polyphosphate, and magnesium polyphosphate.

In addition, preferred examples of stabilizing agents include: phosphoric acid, pyrophosphoric acid, polyphosphoric acid, metaphosphoric acid and salts thereof; citric acid; malic acid; tartaric acid; gluconic acid; fumaric acid; L-ascorbic acid; D-cellobiose; mannitol; inositol; inulin; fructooligosaccharide; glycine; L-alanine; L-leucine; D-leucine; L-glutamine; L-glutamic acid; L-ornithine; ferric chloride; magnesium chloride; calcium chloride; ferrous sulfate; magnesium sulfate; calcium sulfate; sodium sulfate; alminium sulfate; alminium potassium sulfate; and alminium ammonium sulfate. Further preferred examples of stabilizing agents include: phosphoric acid, pyrophosphoric acid, polyphosphoric acid, metaphosphoric acid and salts thereof; citric acid; malic acid; tartaric acid; ferric chloride; magnesium chloride; ferrous sulfate; magnesium sulfate; calcium sulfate; and sodium sulfate. These stabilizing agents may be used alone or in combination of two or more stabilizing agents.

Hitherto, there has been a finding that the stability of SAM in a salt comprising SAM and an acid such as sulfuric acid, hydrochloric acid, or p-toluenesulfonic acid can be improved with the addition of a metal salt such as magnesium sulfate. However, the present inventors have first found that chemical degradation and/or epimerization of (SS)-SAM can be prevented by allowing a metal salt or an ammonium salt to coexist with SAM without forming a salt comprising SAM and an inorganic acid salt, an organic acid salt, or a combined salt described above. Accordingly, it has become possible to stabilize SAM by allowing such metal salt or ammonium salt to coexist with SAM without the use of a strong acid such as sulfuric acid, hydrochloric acid, or p-toluenesulfonic acid. As a result, SAM can be used in a safer form for foods and supplements (health foods and functional foods).

As a metal salt or an ammonium salt to be added, a metal salt is preferable. A metal salt comprising a divalent cation and an acid is more preferable. A metal salt comprising an alkaline-earth metal cation and an acid is further preferable. A salt comprising magnesium or calcium and an acid is particularly preferable. Specific examples of preferable salts include magnesium sulfate, magnesium chloride, calcium sulfate, and calcium chloride. Of these, magnesium sulfate and calcium sulfate are particularly preferable.

The amounts of the above stabilizing agents are not particularly limited. However, the molar ratio of the amount of a stabilizing agent to the amount of SAM contained in microbial cells or microbial extract is generally 0.1:1 or more, preferably 0.5:1 or more, more preferably 1:1 or more, and further preferably 2:1 or more. In addition, the upper limit of the molar ratio is not particularly limited.

Further, when a salt such as a metal salt, an inorganic acid salt, an organic acid salt, or an amino acid salt is used as a stabilizing agent, a compound in the form of a salt may be directly added. Alternatively, an acid and a base that constitute a salt may be separately added. In such case, examples of an acid that constitutes a salt include sulfuric acid, hydrochloric acid, carbonic acid, phosphoric acid, and polyphosphoric acid. In addition, examples of a base that constitutes a salt include ammonia, magnesium hydroxide, calcium hydroxide, sodium hydroxide, and aluminium hydroxide. In such case, an acid and a base used may be used alone or two or more types of acids and bases may be used in combination.

(Definition of Prevention of SAM Degradation and/or (SS)-SAM Epimerization)

According to the present invention, SAM and/or (SS)-SAM stabilization and discontinuation or prevention of SAM degradation and/or (SS)-SAM epimerization are explained as follows. The discontinuation or prevention against “degradation” refers to conditions in which the SAM residual rate is generally 85% or more, preferably 90% or more, more preferably 95% or more, and particularly preferably 99% or more after preservation of dried microbial cells and/or microbial extract containing SAM and/or (SS)-SAM at 40° C. for 3 days. The discontinuation or prevention against “epimerization” refers to conditions in which decrease of the diastereomer ratio is generally 10% d.e. or less, more preferably 5% d.e. or less, further preferably 3% d.e. or less, and particularly preferably 1% d.e. or less. Needless to say, it is preferable that degradation and epimerization can be discontinued/prevented. Decrease of the SAM residual rate and decrease of the diastereomer ratio can be determined by HPLC analysis or the like.

### (Composition)

According to the present invention, in order to improve properties (e.g., fluidity and hygroscopicity) of dried microbial cells and/or microbial extract containing SAM and/or (SS)-SAM and/or to process or use such cells and/or extract in the form of medicine, food (e.g., health food), cosmetic, or the like, a composition comprising dried microbial cells or microbial extract containing SAM and/or (SS)-SAM or a processed product thereof can be formed with the use of a variety of additives according to need. Examples of such additives include, but are not particularly limited to, excipients, disintegrants, lubricants, binders, dyes, anti-agglomerates, absorption promoters, solubilizing agents, stabilizing agents (for stabilizing the state of a composition or a formulation), aroma chemicals, fat and oil, surfactants, fatty acids, non-SAM components, and antioxidants.

Examples of excipients include, but are not particularly limited to, sucrose, lactose, glucose, starch, dextrin, mannitol, crystalline cellulose, calcium phosphate, and calcium sulfate.

Examples of disintegrants include, but are not particularly limited to, starch, agar, calcium citrate, calcium carbonate, sodium hydrogen carbonate, dextrin, crystalline cellulose, carboxymethyl cellulose, tragacanth, and alginic acid.

Examples of lubricants include, but are not particularly limited to, talc, magnesium stearate, polyethylene glycol, silica, and hardened oil.

Examples of binders include, but are not particularly limited to, ethylcellulose, methylcellulose, hydroxypropyl methylcellulose, hydroxypropyl cellulose, tragacanth, shellack, gelatin, pullulan, gum arabic, polyvinyl pyrrolidone, polyvinyl alcohol, polyacrylic acid, polymethacrylic acid, and sorbitol.

Examples of dyes include, but are not particularly limited to, titanium oxide, food dye, colcothar dye, safflower dye, caramel dye, gardenia dye, tar dye, and chlorophyll.

Examples of anti-agglomerants include, but are not particularly limited to, stearic acid, talc, light anhydrous silicic acid, and hydrated silicate dioxide.

Examples of absorption promoters include, but are not particularly limited to, higher alcohols.

Examples of solubilizing agents include, but are not particularly limited to, organic acids such as fumaric acid, succinic acid, and malic acid.

Examples of stabilizing agents (for stabilizing the state of a composition or a formulation) include, but are not particularly limited to, benzoic acid, sodium benzoate, ethyl parahydroxybenzoate, beeswax, hydroxypropyl methylcellulose, and methylcellulose. Herein, stabilizing agents (for stabilizing the state of a composition or a formulation) are additives used to stabilize the state of a composition or a formulation (e.g., fluidity, viscosity, etc.) and can be used regardless of differences from stabilizing agents used to improve SAM stability (against degradation/epimerization).

Examples of aroma chemicals include, but are not particularly limited to, citrus aurantium dulcis (orange) oil, capsicum oil, mustard oil, garlic oil, caraway oil, clove oil, cinnamon oil, cocoa extract, coffee bean extract, ginger oil, spearmint oil, celery seed oil, thyme oil, onion oil, nutmeg oil, parsley seed oil, mint oil, vanilla extract, funnel oil, pennyroyal oil, peppermint oil, eucalyptus oil, lemon oil, rose oil, rosemary oil, almond oil, ajowan oil, anise oil, amyris oil, angelica root oil, ambrette seed oil, estrogen oil, origanum oil, orris root oil, olibanum oil, quassia oil, cascarilla oil, cananga oil, camomile oil, calamus oil, cardamom oil, carrot seed oil, cubeb oil, cumin oil, grapefruit oil, cinnamon oil, cade oil, pepper oil, costus root oil, cognac oil, copaiba oil, coriander oil, labiate oil, musk, juniper berry oil, staranise oil, sage oil, savory oil, geranium oil, tangerin oil, dill oil, neroli oil, true balsam oil, basil oil, birch oil, patchouli oil, palmarosa oil, pimento oil, putitgrain oil, bay leaf oil, bergamot oil, Peru balsam oil, benzoin resin, bois de rose (rosewood) oil, hop oil, boronia absolute, marjoram oil, mandarin oil, myrtle oil, citrus (yuzu) aroma chemicals, lime oil, lavandin oil, lavender oil, rue oil, lemongrass oil, lethionine, lovage oil, laurel leaf oil, and worm wood oil.

Fat and oil may be natural fat and oil, synthetic fat and oil, or processed fat and oil. Dietetically or pharmaceutically acceptable fat and oil are more preferable. Examples of plant fat and oil include coconut oil, palm oil, palm kernel oil, linseed oil, camellia oil, brown rice germ oil, rapeseed oil, rice oil, ground pea oil, corn oil, wheat germ oil, soybean oil, perilla oil, cotton seed oil, sunflower oil (sunflower seed oil), kapok oil, evening primrose oil, shea butter, sal butter, cacao butter, sesame oil, safflower oil, olive oil, avocado oil, poppy oil, arctium lappa seed oil, and peanut oil. Examples of animal fat and oil include lard, milk fat, fish oil, and beef tallow. Further, fat and oil (e.g., hardened oil) obtained by processing the above examples via separation, hydrogenation, or ester exchange can be used. Needless to say, medium-chain triglyceride (MCT) can also be used. Examples of medium-chain triglyceride include, but are not particularly limited to, triglyceride having fatty acid with a carbon number of 6 to 12 and preferably 8 to 12. In addition, a fatty acid portion of triglyceride can also be used. Further, a mixture of the above examples of fat and oil can also be used.

Among the above examples of fat and oil, plant fat and oil, synthetic fat and oil, processed fat and oil, and medium-chain triglyceride are preferable in terms of handleability, odor, or the like.

Examples of surfactants include glycerin fatty acid ester, sucrose fatty acid ester, organic acid monoglyceride, sorbitan fatty acid ester, polyoxyethylene sorbitan fatty acid ester, propylene glycol fatty acid ester, condensed ricinoleic acid glyceride, saponin, and phospholipid.

Examples of glycerin fatty acid ester that can be used include, but are not particularly limited to, monoglycerin fatty acid ester and polyglycerin fatty acid ester. For example, glycerin fatty acid ester comprising glycerin with a degree of polymerization of 1 to 12 and a fatty acid residue with a carbon number of 6 to 22 can be used. In addition, either saturated or unsaturated fatty acid residues can be contained in glycerin fatty acid ester without particular limitation. The number of fatty acid residues of glycerin fatty acid ester differs depending on the degree of polymerization of glycerin or the like. Therefore, it is not particularly limited. The upper limit of such number corresponds to the number of hydroxy groups present in the glycerin structure (i.e., the degree of polymerization of glycerin+2). Glycerin fatty acid ester is not particularly limited regarding fatty acid residues. However, glycerin fatty acid ester having fatty acid residues with carbon numbers of 8 to 22 is preferably used and glycerin fatty acid ester having fatty acid residues with carbon numbers of 8 to 18 is particularly preferably used. Examples of fatty acids that constitute such fatty acid residues include caprylic acid, capric acid, lauric acid, myristic acid, palmitic acid, stearic acid, isostearic acid, oleic acid, linoleic acid, linolenic acid, and behenic acid. In addition, when two or more fatty acid residues are present, fatty acid residues may be the same or different. However, in view of ease of procurement, fatty acid residues are preferably the same.

Sucrose fatty acid ester is not particularly limited. Either saturated or unsaturated fatty acid residues can be contained in sucrose fatty acid ester. However, sucrose fatty acid ester having fatty acid residues with carbon numbers of 8 to 22 is preferably used and sucrose fatty acid ester having fatty acid residues with carbon numbers of 8 to 18 is particularly preferably used. Examples of fatty acids that constitute such fatty acid residues include caprylic acid, capric acid, lauric acid, myristic acid, palmitic acid, stearic acid, isostearic acid, oleic acid, linoleic acid, linolenic acid, and behenic acid. In addition, when two or more fatty acid residues are present, fatty acid residues may be the same or different. However, in view of ease of procurement, fatty acid residues are preferably the same.

Organic acid monoglyceride is not particularly limited. However, examples thereof include acetic acid monoglyceride, citric acid monoglyceride, lactic acid monoglyceride, succinic acid monoglyceride, and tartaric acid monoglyceride such as diacetyl tartaric acid monoglyceride. Herein, fatty acid residues that constitute organic acid monoglyceride are not particularly limited. Examples of fatty acids that constitute such fatty acid residues include caprylic acid, capric acid, lauric acid, myristic acid, palmitic acid, stearic acid, oleic acid, linoleic acid, linolenic acid, and behenic acid. Of these, myristic acid, palmitic acid, stearic acid, and oleic acid are preferable.

Sorbitan fatty acid ester is not particularly limited. Either saturated or unsaturated fatty acid residues can be contained in sorbitan fatty acid ester. However, sorbitan fatty acid ester having fatty acid residues with carbon numbers of 8 to 22 is preferably used and sorbitan fatty acid ester having fatty acid residues with carbon numbers of 8 to 18 is particularly preferably used. Examples of fatty acids that constitute such fatty acid residues include caprylic acid, capric acid, lauric acid, myristic acid, palmitic acid, stearic acid, isostearic acid, oleic acid, linoleic acid, linolenic acid, and behenic acid. Oleic acid is particularly preferable. In addition, when two or more fatty acid residues are present, fatty acid residues may be the same or different. However, in view of ease of procurement, fatty acid residues are preferably the same.

Polyoxyethylene sorbitan fatty acid ester is not particularly limited. Either saturated or unsaturated fatty acid residues can be contained in polyoxyethylene sorbitan fatty acid ester. However, polyoxyethylene sorbitan fatty acid ester having fatty acid residues with carbon numbers of 8 to 22 is preferably used and sorbitan fatty acid ester having fatty acid residues with carbon numbers of 8 to 18 is particularly preferably used. Examples of fatty acids that constitute such fatty acid residues include caprylic acid, capric acid, lauric acid, myristic acid, palmitic acid, stearic acid, isostearic acid, oleic acid, linoleic acid, linolenic acid, and behenic acid. Oleic acid is particularly preferable. In addition, when two or more fatty acid residues are present, fatty acid residues may be the same or different. However, in view of ease of procurement, fatty acid residues are preferably the same.

Propylene glycol fatty acid ester is not particularly limited. Either propylene glycol fatty acid monoester or propylene glycol fatty acid diester can be preferably used. Either saturated or unsaturated fatty acid residues can be contained in propylene glycolfatty acid ester. However, propylene glycol fatty acid ester having fatty acid residues with carbon numbers of 6 to 22 is used, propylene glycol fatty acid ester having fatty acid residues with carbon numbers of 8 to 18 is preferably used, and propylene glycol fatty acid ester having fatty acid residues with carbon numbers of 8 to 12 is more preferably used. Examples of fatty acids that constitute such fatty acid residues include caprylic acid, capric acid, lauric acid, myristic acid, palmitic acid, stearic acid, isostearic acid, oleic acid, linoleic acid, linolenic acid, and behenic acid. Oleic acid is particularly preferable. In addition, in the case of propylene glycol fatty acid diester, fatty acid residues may be the same or different. However, in view of ease of procurement, fatty acid residues are preferably the same.

Any condensed ricinoleic acid poly glyceride can be used regardless of the degree of polymerization of glycerin without particular limitation. However, for example, the degree of polymerization is 2 to 10, preferably 2 or more, more preferably 3 or more, and particularly preferably 4 or more. The upper limit of the degree of polymerization of glycerin is not particularly limited. However, it is generally 10 or less, preferably 8 or less, and more preferably 6 or less.

Examples of saponin include, but are not particularly limited to, enju saponin, quillaja saponin, purified soybean saponin, and yucca saponin.

Examples of phospholipid include, but are not particularly limited to, yolk lecithin, soybean lecithin, phosphatidylcholine, phosphatidylethanolamine, phosphatidylserine, sphingomyelin, dicetylphosphoric acid, phosphatidylglycerol, phosphatidic acid, phosphatidylinositolamine, cardiolipin, ceramide phosphorylethanolamine, ceramide phosphorylglycerol, and mixture thereof. Needless to say, for example, lysophospholipid (lysolecithin) obtained by subjecting such phospholipid to enzyme degradation or the like can also be preferably used.

Examples of fatty acid include, but are not particularly limited to, caproic acid, caprylic acid, lauric acid, myristic acid, palmitic acid, stearic acid, oleic acid, linoleic acid, linolenic acid, and behenic acid.

Examples of active ingredients other than SAM include amino acids, vitamins, minerals, polyphenols, organic acids, sugars, peptides, proteins, coenzyme Q10 (including oxidized coenzyme Q10 and reduced coenzyme Q10), and carotenoid.

Examples of antioxidants include ascorbic acids, tocopherols, vitamin A, β-carotene, sodium hydrogen sulfite, sodium thiosulfate, sodium pyrosulfite, and citric acids. Alternatively, as ascorbic acids and citric acids, fruit juice concentrates (e.g., extract and powder) containing ascorbic acids and citric acids obtained from lemon, orange, grapefruit, and the like can be used.

The above substances may have a plurality functions. For instance, starch may function as an excipient and a disintegrant. Citric acid may function in three different ways as a solubilizing agent, a non-SAM component, and an antioxidant.

### (Packaging/Wrapping)

When the dried microbial cells and microbial extract containing SAM and/or (SS)-SAM of the present invention, a composition containing the same, and a processed product thereof are preserved, it is preferable for them to be wrapped or packaged with a glass, plastic, and/or metal material so as to be placed in an environment isolated from the external environment.

Examples of glass material include soft glass and hard glass. Examples of plastic material include high-density polyethylene, medium-density polyethylene, low-density polyethylene, polypropylene, polyethylene terephthalate, polyvinyl alcohol, polyvinyl chloride, polyvinylidene chloride, and nylon. Needless to say, examples of plastic material also include film obtained by laminating the above plastic material, film obtained by laminating a metal such as aluminium on plastic material (e.g., aluminium laminate), and film obtained by evaporating alumina, silica, or the like on plastic material (e.g., evaporated aluminium film or evaporated silica film).

Examples of metal material include iron, alminium, zinc, nickel, cobalt, copper, tin, titanium, chrome, and alloy of such metal (e.g., stainless-steel or brass). Also, material obtained by combining glass and a metal such as enamel can be used. In order to prevent influx of a gas from the external environment, soft glass, hard glass, aluminium laminate, evaporated aluminium film, evaporated silica film, and metal material are particularly preferable.

The above materials are preferably formed into a bottle, bag, can, drum, box, or the like for wrapping/packaging of the composition of the present invention. It is also possible to carry out PTP wrapping, three-side seal wrapping, four-side seal wrapping, pillow wrapping, strip wrapping, aluminium-forming wrapping, and stick wrapping with the use of the above materials. When a material having relatively high gas permeability (e.g., polyethylene) is used, it is preferable to repeat wrapping/packaging at least twice. In such case, it is particularly preferable to use a material having relatively high gas barrier properties and moisture-proof properties such as aluminium laminate, evaporated alumina or silica film, glass, and metal. After wrapping/packaging, the obtained product can be transferred or stored in an iron steel drum, a resin drum, a fiber drum, a cardboard case, or the like according to need.

For wrapping/packaging described above, a desiccant or a deoxidant can be used in combination during wrapping or packaging. Particularly preferably, a moisture-proof agent is used in combination. Examples of moisture-proof agent include silica gel, calcium chloride, and synthetic zeolite.

In addition, after wrapping/packaging of the dried microbial cells and microbial extract containing (SS)-SAM, the dried microbial cells and microbial extract containing (SS)-SAM may be subjected to maturing.

### (Processing)

The dried microbial cells and microbial extract containing SAM and/or (SS)-SAM obtained in the present invention can be directly used in or processed into a food, a food with nutrient function claims, a food for specified health use, a nutritious supplement, a nutrient, a beverage, a veterinary drug, a feed, a cosmetic, a quasi drug, a pharmaceutical product, or a therapeutic or prophylactic agent. The processed form of the composition of the present invention is an oral administration form such as capsules (e.g., microcapsules, hard capsules, or soft capsules (preferably microcapsules or soft capsule)), tablets, a powder, chewable tablets, granules, pills, a syrup, or a beverage. Also, it can be processed and used in the form of cream, suppository, tooth paste, or the like.

When processing the dried microbial cells and microbial extract containing SAM and/or (SS)-SAM of the present invention into the above form, it is possible to the aforementioned add additives (e.g., excipients, disintegrants, lubricants, binders, dyes, anti-agglomerant, absorption promoters, solubilizing agents, stabilizing agents, aroma chemicals, fat and oil, surfactants, fatty acids, non-SAM components, and antioxidants). It is preferable to add additives in order to obtain desired properties.

Examples of preferable processed forms include capsules, tablets, powders, granules, chewable tablets, and pills. Tablets, chewable tablets, and capsules are particularly preferable.

In the case of the form of capsules, base materials for capsules are not particularly limited. In addition to gelatins obtained from bovine bones, bovine skin, pig skin, and fish skin, other base materials (e.g., products that can be used as food additives, including seaweed-derived products (e.g., carrageenan and alginic acid), plant seed-derived products (e.g., locust bean gum and guar gum), thickening stabilizers such as plant-secretion-derived products (e.g., gum Arabic), and production agents such as celluloses) can be used.

In addition, in the cases of a formulation in the form of tablets, chewable tablets, granules, or a powder, it is preferable to coat the above formulation with an oil-soluble coating medium and/or a water soluble coating medium and preferably a dietetically acceptable oil-soluble coating medium and/or a water soluble coating medium in order to prevent chemical degradation and/or epimerization of SAM and/or (SS)-SAM in the formulation.

Examples of the above oil-soluble coating medium include: fatty acid sugar ester; shellack or cellulose derivatives; fatty acids and ester derivatives thereof; and fat and oil and ester derivatives thereof. Preferably, shellack and cellulose derivatives are used. More preferably, shellack is used.

Examples of the above water soluble coating medium include gelatin, sugar, gum arabic, fatty acid sugar ester, tragacanth, pectin, pullulan, dried albumen, milk, curdlan, cellulose derivatives, casein, casein compounds, starch, Zein, and yeast cell wall. Preferably, gelatin, sugar, gum arabic, pullulan, cellulose derivatives, Zein, and yeast cell wall are used. More preferably gelatin, sugar, cellulose derivatives, and yeast cell wall are used. Further preferably, gelatin and yeast cell wall are used. Particularly preferably yeast cell wall is used.

Examples of the above sugar include: monosaccharides and disaccharides such as sucrose (e.g., purified saccharose or saccharose), fructose, glucose, lactose, and trehalose; sugar alcohols such as erythritol, mannitol, sorbitol, xylitol, maltitol, glutinous starch syrup of powdered reduced maltose, and reduced lactose; and polysaccharides such as dextrin and maltodextrin.

In addition, the dietetically acceptable coating medium herein defined refers to an arbitrary nontoxic coating medium that is used for coating of the above formulation in the art. Such coating medium is not particularly limited. However, examples thereof include gelatin, sugar, gum arabic, pullulan, cellulose derivatives, yeast cell wall, and shellack.

Needless to say, such coating medium can be used in the form of a mixture of two or more substances. In addition, coating with each substance can be carried out twice or more.

For instance, in order to improve moisture-proof properties and water resistance of the above formulation, the formulation can be coated with an oil-soluble coating medium and then further coated with a water soluble coating medium. Alternatively, the formulation can be coated with a water soluble coating medium and then further coated with an oil-soluble coating medium.

When a composition containing the dried microbial cells or microbial extract containing SAM and/or (SS)-SAM described above is prepared, or when the composition is processed into a desired form such as capsules, tablets, or a powder, the relative humidity of a surrounding environment is preferably at a minimum level. Such humidity is generally 70% RH or less, preferably 60% RH or less, more preferably 50% RH or less, further preferably 30% RH or less, and particularly preferably 20% RH or less.

In addition, if the temperature of a surrounding environment is high during preparation and processing of the composition, SAM degradation is promoted. Therefore, the temperature of a surrounding environment should be generally 50° C. or less, preferably 40° C. or less, more preferably 35° C. or less, further preferably 25° C. or less, and particularly preferably 20° C. or less. The lower limit of the temperature is not particularly limited.

During preparation and processing described above, the time required for exposure of the composition containing dried microbial cells and microbial extract containing SAM and/or (SS)-SAM to a humidity of 20% RH or more and a temperature of 20° C. or more is not particularly limited. However, it is generally 96 hours or less, preferably 72 hours or less, more preferably 48 hours or less, and particularly preferably 24 hours or less. In addition, the above surrounding environment refers to, for example, a working environment such as the environment of a room in which operations are carried out for preparation or processing of the composition containing dried microbial cells and microbial extract containing SAM and/or (SS)-SAM.

In addition, as a result of preparation or processing of the composition described above, fat solubility, controlled release properties, high absorbability, a function of preventing further SAM degradation can be imparted.

Further, the dried microbial cells and microbial extract containing SAM and/or (SS)-SAM of the present invention and the above preparation may be added to a general food product for use. Examples of such food products include: dairy products such as milk, milk beverages, cheese, milk powder formula, ice cream, and yoghurt; beverages such as juice, lactic acid beverages, tea, and coffee; sweets such as chocolate, cookie, biscuits, candy, Japanese sweets, rice sweets, cake, pie, and pudding; wheat products bread and noodle; rice products such as risotto and boiled rice; and seasonings such as soy sauce, miss, mayonnaise, and dressing. Needless to say, such food products may be processed seafood products, processed agriculture products, and processed livestock products. In addition, they can be used in other food forms.

In view of stability of dried microbial cell or microbial extract containing (SS)-SAM, in a preferred embodiment, the aforementioned wrapping/packaging is carried out in a manner such that the formulation is isolated from the external environment even after processing.

According to the present invention, dried microbial cells or microbial extract containing (SS)-SAM is subjected to maturing treatment such that (SS)-SAM degradation and epimerization can be significantly prevented or discontinued. Accordingly, dried microbial cells or microbial extract containing stabilized (SS)-SAM can be obtained without the use of the excessive amounts of additives.

According to the present invention, a composition comprising dried microbial cells or microbial extract containing SAM is prepared or processed in a specific environment without the need of high cost, complicated procedures, or a particular facility, allowing prevention of SAM degradation.

### EXAMPLES

The present invention is hereafter described in greater detail with reference to the following examples, although the technical scope of the present invention is not limited thereto.

### Reference Example

### Culture of a Microorganism Containing SAM and/or (SS)-SAM

A medium containing sucrose (150 g/L), ethanol (18 g/L), yeast extract (10 g/L), L-methionine (10 g/L), urea (18 g/L), glycine (2 g/L), potassium dihydrogen phosphate (4 g/L), magnesium sulfate heptahydrate (0.2 g/L), biotin (2 mg/L), calcium chloride dehydrate (0.2 g/L), zinc sulfate heptahydrate (10 mg/L), ferrous sulfate heptahydrate (5 mg/L), manganese sulfate tetrahydrate (5 mg/L), cobalt chloride hexahydrate (0.2 mg/L), copper sulfate pentahydrate (0.1 mg/L), and potassium iodide (0.1 mg/L) was inoculated with the Saccharomyces cerevisiae K-6 strain. Culture was carried out for 4 days during aeration and agitation in a 5-L jar incubator at 30° C. Yeast cells were collected from the obtained culture solution by centrifugation. Deionized water was added to the yeast cells to result in equal amounts of the culture solution, followed by suspension. Then, a washing operation was carried out twice to collect yeast cells by centrifugation. Thus, yeast cells containing (SS)-SAM were obtained.

The (SS)-SAM content in the obtained yeast cells was 12% by weight based on the dry weight of the yeast cells. In addition, the (SS)-SAM content in the yeast cells and the diastereomer ratio were analyzed via high performance liquid chromatography in accordance with the procedures described below.

### [HPLC Analysis Conditions]

Column: Develosil ODS-HG5 (4.6 mm φ×250 mm, Nomura Chemical Co., Ltd.); eluent: 60 mM potassium phosphate buffer (pH 2.5) and 8 mM 1-decanesulfonatesodium:methanol=53:47; flow rate: 0.5 ml/minute; column temperature: 25° C.; measurement wavelength: 254 nm.

### Example 1

### Preparation of Dried Microbial Cells of a Microorganism Containing (SS)-SAM

The yeast cells obtained in the Reference Example were suspended in deionized water in one-sixth (⅙) of the amount of the culture solution, followed by freezing at −80° C. and lyophilization. The obtained dried yeast cells were matured in the presence of silica gel at 40° C. for 7 days. Thus, dried yeast cells containing (SS)-SAM were obtained.

Table 1 lists the results obtained by preserving the resulting dried yeast cells containing (SS)-SAM at 40° C. and determining the (SS)-SAM residual rate and the rate of decrease of the diastereomer ratio over time.

### Comparative Example 1

Dried yeast cells containing (SS)-SAM were obtained in the same manner as that used in Example 1, except that the maturing operation following lyophilization was not carried out. Table 1 lists the results obtained by preserving the resulting dried yeast cells containing (SS)-SAM at 40° C., determining the (SS)-SAM residual rate and the rate of decrease of the diastereomer ratio over time, and comparing the results with the results obtained in Example 1.

TABLE 1

| | Example 1 | | Comparative Example 1 | |
|---|---|---|---|---|
| | (matured) | | (non-matured) | |
| | | Rate of decrease | (SS)- | Rate of decrease |
| Preservation | (SS)-SAM | of the (SS)-SAM | SAM | of the (SS)-SAM |
| period | residual | diastereomer | residual | diastereomer |
| (day) | rate (%) | ratio (% d.e.) | rate (%) | ratio (% d.e.) |
| 0 | 100.0 | 0.0 | 100.0 | 0.0 |
| 3 | 92.9 | 2.3 | 68.8 | 16.2 |
| 7 | 84.8 | 4.7 | 58.4 | 21.1 |

As shown in table 1, (SS)-SAM contained in dried yeast cells subjected to maturing treatment was significantly more stable against chemical degradation and epimerization than (SS)-SAM contained in untreated dried yeast cells.

### Example 2

### Preparation of Microbial Extract Containing (SS)-SAM

The yeast cells obtained in the Reference Example were suspended in deionized water in one-sixth (⅙) of the amount of the culture solution, followed by pressure disruption of microbial cells (with the use of HOMOGENIZER Rannie 2000; APV HOMOGENIZER GROUP) (disruption conditions: 100 MPa; passing: 6 passages). Then, the pH was adjusted to 4 with 30% NaOH. Undissolved matter was removed from the resultant via centrifugation (5640 G, 20 minutes) such that a yeast cell extract was obtained. Dextrin (20% by weight of the dry weight of the yeast cell extract) was added to the obtained yeast cell extract, followed by freezing at −80° C. and lyophilization. The obtained dried yeast cell extract was matured in the presence of silica gel at 40° C. for 7 days. Accordingly, a dried yeast cell extract containing (SS)-SAM was obtained.

Table 2 lists the results obtained by preserving the resulting dried yeast cell extract containing (SS)-SAM at 40° C. and determining the (SS)-SAM residual rate and the rate of decrease of the diastereomer ratio over time.

### Comparative Example 2

Dried yeast cell extract containing (SS)-SAM was obtained in the same manner as that used in Example 2 except that the maturing operation following lyophilization was not carried out. Table 2 lists the results obtained by preserving the resulting dried yeast cell extract containing (SS)-SAM at 40° C., determining the (SS)-SAM residual rate and the rate of decrease of the diastereomer ratio over time, and comparing the results with the results obtained in Example 2.

TABLE 2

| | Example 2 | | Comparative Example 2 | |
|---|---|---|---|---|
| | (matured) | | (non-matured) | |
| | | Rate of decrease | (SS)- | Rate of decrease |
| Preservation | (SS)-SAM | of the (SS)-SAM | SAM | of the (SS)-SAM |
| period | residual | diastereomer | residual | diastereomer |
| (day) | rate (%) | ratio (% d.e.) | rate (%) | ratio (% d.e.) |
| 0 | 100.0 | 0.0 | 100.0 | 0.0 |
| 3 | 95.8 | 1.0 | 79.5 | 13.4 |
| 7 | 92.5 | 1.9 | 68.1 | 17.4 |

As shown in table 2, (SS)-SAM contained in dried yeast cell extract subjected to maturing treatment was significantly more stable against chemical degradation and epimerization than (SS)-SAM contained in untreated dried yeast cell extract.

### Example 3

### Stabilizing Agent

The yeast cells obtained in the Reference Example were suspended in deionized water in one-sixth (⅙) of the amount of the culture solution. Each of the compounds listed in table 3 (30% by weight in terms of dry weight) was separately added to the resultant, followed by freezing at −80° C. and lyophilization. Thus, dried yeast cells containing SAM were obtained.

Table 3 lists the results obtained by preserving the obtained dried yeast cells containing SAM at 25° C. for 7 days and determining the SAM residual rate.

TABLE 3

| | SAM | | SAM |
|---|---|---|---|
| | residual | | residual |
| Compound name | rate (%) | Compound name | rate (%) |
| Compound-free | 83.6 | Glucono-δ-lactone | 100.7 |
| Citric acid | 96.6 | Glycine | 97.5 |
| L-tartaric acid | 88.1 | L-alanine | 86.9 |
| DL-malic acid | 99.1 | L-valine | 89.7 |
| L-ascorbic acid | 90.0 | D-leucine | 91.9 |
| Sodium gluconate | 89.1 | L-threonine | 92.0 |
| Nicotinic acid | 102.3 | L-methionine | 100.9 |
| Nicotinic-acid amide | 97.3 | L-glutamine | 94.5 |
| Galactose | 99.7 | L-ornithine hydrochloride | 98.2 |
| Sucrose | 93.8 | DL-α-amino-n-butyrate | 96.8 |
| D-cellobiose | 93.1 | thiamine hydrochloride | 99.5 |
| Cellulose | 92.0 | Phosphoric acid | 89.3 |
| N-acetylglucosamine | 97.6 | Ammonium dihydrogen | 93.6 |
| | | phosphate | |
| L-sorbose | 88.4 | Ammonium chloride | 95.6 |
| Inulin | 93.1 | Ferric chloride | 95.7 |
| | | hexahydrate | |
| Glycogen | 94.6 | Magnesium chloride | 101.2 |
| | | hexahydrate | |
| Dulcitol | 92.2 | Ammonium sulfate | 87.5 |
| Mannitol | 86.8 | Ferrous sulfate | 90.4 |
| | | heptahydrate | |
| Sorbitol | 98.2 | Magnesium sulfate | 101.6 |
| Inositol | 96.2 | Manganese sulfate | 95.0 |
| Fructo-oligosaccharide | 85.9 | Nickel(II) sulfate | 96.5 |
| Isomalto-oligosaccharide | 84.9 | Calcium chloride | 93.7 |
| Lactitol | 91.3 | Zinc sulfate | 94.3 |
| Galactitol | 90.7 | Copper sulfate | 94.2 |

### Example 4

### Bioabsorbability of Dried Yeast Cells Containing (SS)-SAM

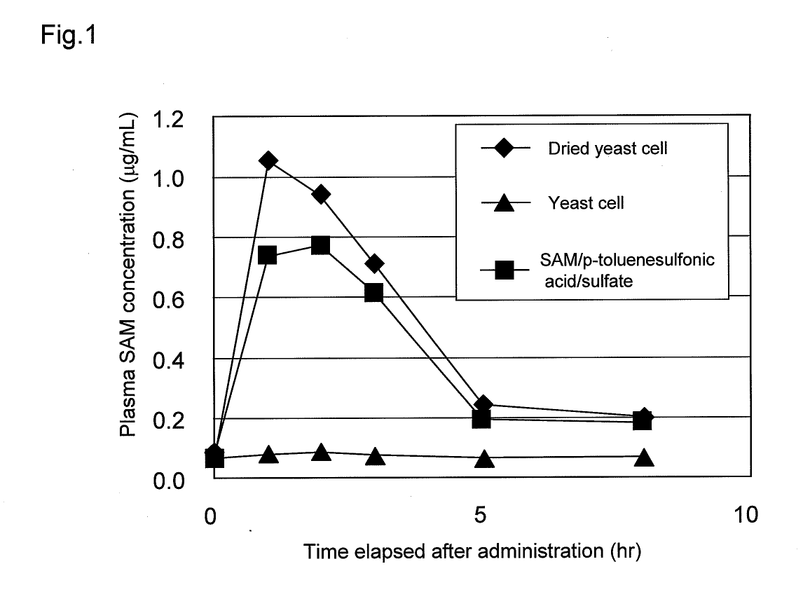

The dried yeast cells containing (SS)-SAM obtained in Example 1 were suspended in distilled water to result in an SAM concentration of 20 g/L. The suspension was orally administered to SD rats (male, 17 weeks old) at a dose of 5 ml/kg b.w. In addition, yeast cells obtained in Example 1 and SAM/p-toluenesulfonic acid/sulfate (tradename: Gumbaral) were suspended or dissolved in distilled water to result in the above SAM concentration. The resultant was orally administered to rats in the same manner such that comparative control examples were obtained. Before administration and 1, 2, 3, 5, or 8 hours after administration, blood was collected from the jugular vein without any anesthesia and with the use of heparin as an anticoagulant. The plasma components were collected via centrifugation. A 0.4 g/L trichloroacetic acid aqueous solution (40 μL) was added to the obtained plasma (200 μL), followed by mixing and centrifugation. The SAM concentration in the obtained supernatant was analyzed by HPLC. FIG. 1 and table 4 show the results. As shown in FIG. 1 and table 4, bioabsorbability in the case of oral administration of dried yeast cells containing (SS)-SAM was significantly higher than that in the case of oral administration of undried yeast cells. In addition, it was comparable to that in the case of oral administration of SAM/p-toluenesulfonic acid/sulfate.

TABLE 4

| | Maximum SAM | Area under SAM |
|---|---|---|
| | concentration in | concentration curve |
| | plasma | (AUC) |
| | (μg/ml) | (μg/ml × h)(*) |
| Dried yeast cells | 1.054 | 4.033 |
| Yeast cells | 0.085 | 0.567 |
| SAM/p-toluenesulfonic | 0.771 | 3.200 |
| acid/sulfate | | |

(*)AUC at 8 hours after administration

### Example 5

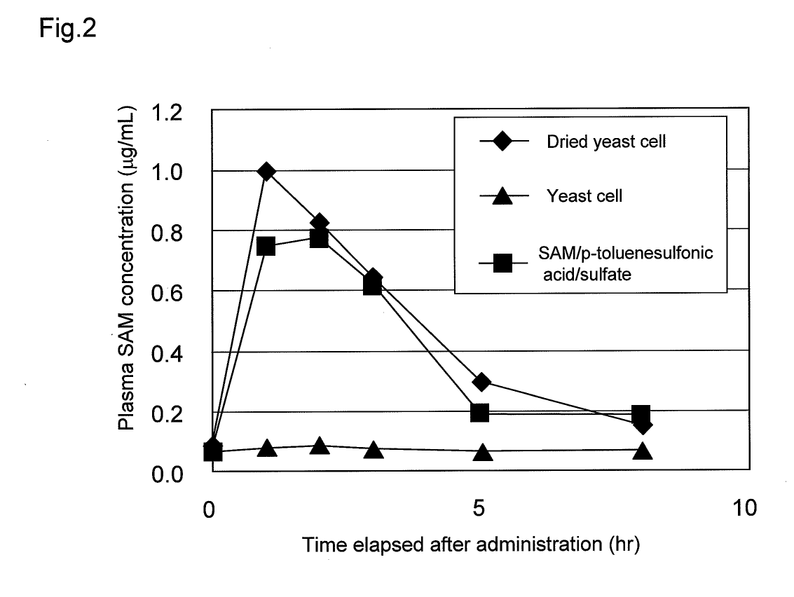

The yeast cells obtained in the Reference Example were suspended in deionized water in one half of the amount of the culture solution, followed by spray drying at an inlet temperature of 185° C. and an outlet temperature of 85° C. The obtained dried yeast cells were matured in the presence of silica gel at 40° C. for 7 days. Thus, dried yeast cells containing (SS)-SAM were obtained. The obtained dried yeast cells containing (SS)-SAM were suspended in distilled water to result in an SAM concentration of 20 g/L. The resultant was orally administered to SD rats (male, 17 weeks old) at a dose of 5 ml/kg b.w. In addition, yeast cells obtained in Example 1 and SAM/p-toluenesulfonic acid/sulfate (tradename: Gumbaral) were suspended or dissolved in distilled water to result in the above SAM concentration. The resultant was orally administered to rats in the same manner such that comparative control examples were obtained. Before administration and 1, 2, 3, 5, or 8 hours after administration, blood collection was carried out from the jugular vein without any anesthesia and with the use of heparin as an anticoagulant. The plasma components were collected via centrifugation. A 0.4 g/L trichloroacetic acid aqueous solution (40 μL) was added to the obtained plasma (200 μl), followed by mixing and centrifugation. The SAM concentration in the obtained supernatant was analyzed by HPLC. FIG. 2 and table 5 show the results. As shown in FIG. 2 and table 5, bioabsorbability in the case of oral administration of dried yeast cells containing (SS)-SAM was significantly higher than that in the case of oral administration of undried yeast cells. In addition, it was comparable to that in the case of oral administration of SAM/p-toluenesulfonic acid/sulfate.

TABLE 5

| | Maximum SAM | Area under SAM |
|---|---|---|
| | concentration in | concentration curve |
| | plasma | (AUC) |
| | (μg/ml) | (μg/ml × h)(*) |
| Dried yeast cells | 0.990 | 3.766 |
| Yeast cells | 0.085 | 0.567 |
| SAM/p-toluenesulfonic | 0.771 | 3.200 |
| acid/sulfate | | |

(*)AUC at 8 hours after administration

### Example 6

### Stability of Dried Yeast Cell Extract Containing SAM

Yeast cells obtained in the Reference Example were suspended in deionized water in one-sixth (⅙) of the amount of the culture solution, followed by pressure disruption (with the use of HOMOGENIZER Rannie 2000; APV HOMOGENIZER GROUP) of microbial cells (disruption conditions: 100 MPa; passing: 6 passages). Then, the pH was adjusted to 4 with 30% NaOH. Undissolved matter was removed from the resultant via centrifugation (5640 G, 20 minutes) such that a yeast cell extract was obtained. Dextrin (20% by weight of the dry weight of the dried yeast cell extract) was added to the obtained yeast cell extract, followed by freezing at −80° C. and lyophilization. The obtained dried yeast cell extract was preserved at a different relative humidity at 25° C. The SAM residual rate and the rate of decrease of the diastereomer ratio were determined over time. Table 6 lists the results. In addition, the moisture content of the obtained dried yeast cell extract was 6%. Herein, the relative humidity was measured with a CTH-201 hygrometer (CUSTOM).

TABLE 6

| | | | | Rate of decrease of the SAM | | |
|---|---|---|---|---|---|---|
| Preservation | SAM residual rate (%) | | | diastereomer ratio (% d.e.) | | |
| period | Humidity: | Humidity: | Humidity | Humidity: | Humidity: | Humidity |
| (Hr) | 32% RH | 50% RH | 60% RH | 32% RH | 50% RH | 60% RH |
| 1 | 99.9 | 99.9 | 99.9 | 0.3 | 0.2 | 0.3 |
| 2 | 99.8 | 99.8 | 99.8 | 0.5 | 0.5 | 0.5 |
| 3 | 99.7 | 99.7 | 99.7 | 0.7 | 0.7 | 0.8 |
| 4 | 99.7 | 99.7 | 99.6 | 0.5 | 0.7 | 0.8 |

Table 6 lists the stability of SAM contained in the dried yeast cell extract under different relative humidity conditions for the relevant period. As shown in table 6, SAM contained in the dried yeast cell extract was highly stable against chemical degradation under different relative humidity conditions.

### Example 7

Table 7 lists the results obtained by preserving dried yeast cell extract that had been preserved under different relative humidity conditions in Example 6 in the presence of silica gel at 40° C. for 8 days and determining the residual rate of SAM contained in the dried yeast cell extract.

TABLE 7

| | | | | Rate of decrease of the SAM | | |
|---|---|---|---|---|---|---|
| Preservation | SAM residual rate (%) | | | diastereomer ratio (% d.e.) | | |
| period | Humidity: | Humidity: | Humidity | Humidity: | Humidity: | Humidity |
| (Hr) | 32% RH | 50% RH | 60% RH | 32% RH | 50% RH | 60% RH |
| 0 | 84.6 | | | 23.7 | | |
| 1 | 87.1 | 84.6 | 85.0 | 25.5 | 23.6 | 23.5 |
| 2 | 84.7 | 84.8 | 85.0 | 23.1 | 23.3 | 23.0 |
| 3 | 84.7 | 85.2 | 85.6 | 22.6 | 22.3 | 22.4 |
| 4 | 85.5 | 84.2 | 84.6 | 22.9 | 22.5 | 22.3 |

There were no differences in SAM residual rate between dried yeast cell extract that had not been preserved under different relative humidity conditions and dried yeast cell extract that had been preserved under different relative humidity conditions. Therefore, the dried yeast cell extract was found to be highly stable against chemical degradation.

### Example 8

### Stability of a Microbial Extract Containing SAM

Table 8 lists the results obtained by preserving dried yeast cell extract obtained in Example 6 at a relative humidity of 60% RH at 25° C. and determining the SAM residual rate over time.

TABLE 8

| | | Rate of decrease |
|---|---|---|
| Preservation | SAM | of the SAM |
| period | residual | diastereomer |
| (Hr) | rate (%) | ratio (% d.e.) |
| 8.5 | 99.7 | 1.0 |
| 24 | 99.3 | 2.5 |
| 48 | 98.7 | 4.1 |

As shown in table 8, SAM contained in the dried yeast cell extract was highly stable against chemical degradation at a relative humidity of 60% RH.

### Example 9

Table 9 lists the results obtained by preserving dried yeast cell extract that had been preserved under different relative humidity conditions in Example 8 in the presence of silica gel at 40° C. for 14 days and determining the SAM residual rate.

TABLE 9

| Preservation | | Rate of decrease |
|---|---|---|
| period in | SAM | of the SAM |
| Example 8 | residual | diastereomer |
| (Hr) | rate (%) | ratio (% d.e.) |
| 0 | 91.4 | 24.6 |
| 8.5 | 92.4 | 18.5 |
| 24 | 91.1 | 17.5 |
| 48 | 87.2 | 16.9 |

There were substantially no differences in SAM residual rate between dried yeast cell extract that had not been preserved under different relative humidity conditions and dried yeast cell extract that had been preserved under different relative humidity conditions. Therefore, the dried yeast cell extract was found to be highly stable against chemical degradation.

### Example 10

### Preparation of Dried Microbial Cells Containing SAM

Yeast cells obtained in the Reference Example were suspended in deionized water in one-sixth (⅙) of the amount of the culture solution, followed by freezing at −80° C. and lyophilization. Thus, dried yeast cells containing SAM were obtained.

All of the Formulation Examples described below were conducted under conditions of a relative humidity of 50% RH or less and a temperature of 25° C. or less. In addition, the relative humidity was determined with the use of a CTH-201 hygrometer (CUSTOM).

### Formulation Example 1

### Soft Capsules

Dried yeast cell extract containing (SS)-SAM obtained in Example 2 or dried yeast cell extract containing SAM obtained in Example 6 was added to a mixture of rapeseed oil, hardened oil, beeswax, and lecithin. Then, soft gelatin capsules comprising the following components, including dried yeast cell extract containing (SS)-SAM or dried yeast cell extract containing SAM, were obtained via a general method.

Dried yeast cell extract containing (SS)-SAM or dried yeast cell extract containing SAM: 30.0% by weight

Rapeseed oil: 55.0% by weight

Hardened oil: 7.0% by weight

Beeswax: 6.0% by weight

Lecithin: 2.0% by weight

The obtained soft capsules were subjected to three-side seal wrapping with the use of an aluminium laminate.

### Formulation Example 2

### Soft Capsules

Dried yeast cell extract containing (SS)-SAM obtained in Example 2 or dried yeast cell extract containing SAM obtained in Example 6 was added to a mixture of medium-chain triglyceride (MCT), hardened oil, beeswax, and lecithin. Then, carrageenan/starch soft capsules comprising the following components, including dried yeast cell extract containing (SS)-SAM or dried yeast cell extract containing SAM, were obtained by a general method.

Dried yeast cell extract containing (SS)-SAM or dried yeast cell extract containing SAM: 25.0% by weight

Medium-chain triglyceride (MCT): 60.0% by weight

Hardened oil: 8.0% by weight

Beeswax: 5.0% by weight

Lecithin: 2.0% by weight

The obtained soft capsules were subjected to three-side seal wrapping with the use of an aluminium laminate.

### Formulation Example 3

### Hard Capsules

Dried yeast cells containing (SS)-SAM obtained in Example 1 or dried yeast cells containing SAM obtained in Example 10, lactose, and magnesium stearate were mixed together. The obtained mixed powder was granulated with a sieve. Then, hard gelatin capsules comprising the following components, including dried yeast cells containing (SS)-SAM or dried yeast cells containing SAM, were obtained by a general method.

Dried yeast cells containing (SS)-SAM or dried yeast cells containing SAM: 60.0% by weight

Lactose: 39.0% by weight

Magnesium stearate: 1.0% by weight

The obtained hard capsules were placed in a hard glass bottle with silica gel. The bottle was sealed.

### Formulation Example 4

### Chewable Tablets

Dried yeast cells containing (SS)-SAM obtained in Example 1 or dried yeast cells containing SAM obtained in Example 10, cornstarch, and sucrose were mixed together. Further, magnesium stearate was added thereto, followed by mixing. The thus obtained mixed powder was granulated with a sieve. Then, the obtained granulate powder was formed into tablets with the use of a rotary tabletting machine. Accordingly, chewable tablets comprising the following components, including dried yeast cells containing (SS)-SAM or dried yeast cells containing SAM, were obtained.

Dried yeast cells containing (SS)-SAM or dried yeast cells containing SAM: 52.0% by weight

Cornstarch: 5.0% by weight

Sucrose: 42.0% by weight

Magnesium stearate: 1.0% by weight

The obtained chewable tablets were subjected to PTP wrapping and the resulting wrapped products were further subjected to pillow wrapping with an aluminium wrapper.

### Formulation Example 5

### Tablets

Dried yeast cell extract containing (SS)-SAM obtained in Example 2 or dried yeast cell extract containing SAM obtained in Example 6 and crystalline cellulose (Avicel) were mixed together. Further, magnesium stearate was added thereto, followed by mixing. The obtained mixed powder was granulated with a sieve. The thus obtained granulate powder was formed into tablets with a rotary tabletting machine. Accordingly, tablets comprising the following components, including dried yeast cell extract containing (SS)-SAM or dried yeast cell extract containing SAM, were obtained.

Dried yeast cell extract containing (SS)-SAM or dried yeast cell extract containing SAM: 49.0% by weight

Crystalline cellulose (Avicel): 50.0% by weight

Magnesium stearate: 1.0% by weight

The obtained tablets were placed in a hard glass bottle with silica gel. The bottle was sealed.

### Formulation Example 6

### Powder

Dried yeast cell extract containing (SS)-SAM obtained in Example 2 or dried yeast cell extract containing SAM obtained in Example 6, lactose, and cornstarch were mixed together. Thus, a powder comprising the following components, including the dried yeast cell extract containing (SS)-SAM or the dried yeast cell extract containing SAM, was obtained

Dried yeast cell extract containing (SS)-SAM or dried yeast cell extract containing SAM: 20.0% by weight

Lactose: 70.0% by weight

Cornstarch: 10.0% by weight

The obtained powder was subjected to stick wrapping with the use of an evaporated aluminium film.

### Formulation Example 7

### Coated Tablets

A shellack-ethanol solution (Gifu Shellac Manufacturing Co., Ltd.) was sprayed over the chewable tablets obtained in Formulation Example 4, followed by drying. Thus, shellack-coated chewable tablets (coated tablets) containing dried yeast cells containing (SS)-SAM or dried yeast cells containing SAM were produced. The obtained chewable tablets were placed in a hard glass bottle with silica gel. The bottle was sealed.

### Formulation Example 8

### Coated Tablets

A solution comprising purified water (450 g) and hydroxypropyl methylcellulose (Shin-Etsu Chemical Co., Ltd.; Metolose 90SH-04) (50 g) was sprayed over the tablets containing a dried yeast cell extract containing (SS)-SAM or dried yeast cell extract containing SAM obtained in Formulation Example 5, followed by drying. Thus, hydroxypropyl-methylcellulose-coated tablets (coated tablets) containing a dried yeast cell extract containing (SS)-SAM or dried yeast cell extract containing SAM were produced. The obtained chewable tablets were placed in a hard glass bottle with silica gel. The bottle was sealed.

1-29. (canceled)

30. A method for preventing (SS)-S-adenosyl-L-methionine degradation and/or epimerization, comprising adding a metal salt and/or an ammonium salt to microbial cells or microbial extract containing (SS)-S-adenosyl-L-methionine so as to allow the salt to coexist therewith.

31. The method according to claim 30, wherein the metal salt and/or ammonium salt is at least one compound selected from the group consisting of ammonium chloride, ferric chloride, magnesium chloride, calcium chloride, aluminium chloride, copper sulfate, ferrous sulfate, magnesium sulfate, calcium sulfate, manganese sulfate, nickel sulfate, zinc sulfate, copper sulfate, ammonium sulfate, sodium sulfate, aluminium sulfate, aluminium potassium sulfate, aluminium ammonium sulfate, ammonium carbonate, and sodium carbonate.

32. The method according to claim 30, wherein the molar ratio of the added metal salt and/or ammonium salt to (SS)-S-adenosyl-L-methionine is from 0.1:1 to 20:1.

33. The method according to claim 30, wherein the (SS)-S-adenosyl-L-methionine in the microbial cells or microbial extract is free (SS)-S-adenosyl-L-methionine.

34. A method for producing dried microbial cells or microbial extract containing (SS)-S-adenosyl-L-methionine, comprising culturing a microorganism capable of producing (SS)-S-adenosyl-L-methionine and drying the obtained microbial cells or microbial extract, wherein an inorganic acid salt is added to microbial cells or microbial extract before drying and retained during drying.

35. The method according to claim 34, wherein the inorganic acid salt is at least one metal salt and/or ammonium salt selected from the group consisting of ammonium chloride, ferric chloride, magnesium chloride, calcium chloride, aluminium chloride, copper sulfate, ferrous sulfate, magnesium sulfate, calcium sulfate, manganese sulfate, nickel sulfate, zinc sulfate, copper sulfate, ammonium sulfate, sodium sulfate, aluminium sulfate, aluminium potassium sulfate, aluminium ammonium sulfate, ammonium carbonate, and sodium carbonate.

36. The method according to claim 34, wherein the microbial cells or microbial extract are not exposed to an environment in which the temperature exceeds 20° C. for 24 hours or more while the moisture content in the microbial cells or microbial extract exceeds 10% in the drying step.

37. The method according to claim 34, wherein drying is carried out by spray drying or lyophilization.

38. The method according to claim 34, wherein the microbial extract is a microbial extract obtained by disrupting cultured microbial cells and, if necessary, removing solid matter.

39. The method according to claim 34, which further comprises maturing after drying.

40. The method according to claim 39, wherein maturing is carried out by placing dried microbial cells or microbial extract containing (SS)-S-adenosyl-L-methionine in a specific environment isolated from the external environment, and wherein the specific environment is achieved through any one or a combination of the following: creation of a depressurized state, substitution with a dried inert gas, and the use of a desiccant.

41. The method according to claim 39, wherein the relative humidity during maturing is 30% RH or less.

42. The method according to claim 39, wherein the time required for maturing is 2 hours or more.

43. A method for producing a formulation containing (SS)-S-adenosyl-L-methionine, comprising processing the dried microbial cells or microbial extract containing (SS)-S-adenosyl-L-methionine, which is obtained by the production method according to claim 34, into a formulation.

44. The method according to claim 43, wherein the moisture content of the dried microbial cells or microbial extract to be processed is 10% or less.

45. The method according to claim 43, wherein the relative humidity during processing is 70% RH or less.

46. The method according to claim 43, wherein the temperature during processing is 50° C. or less.

47. The method according to claim 43, wherein the exposure time of the dried microbial cells or microbial extract to a humidity of 20% RH or more and a temperature of 20° C. or more is 96 hours or less in the processing step.

48. The method according to claim 43, wherein the formulation is a formulation for oral administration in the form of capsules, tablets, a powder, chewable tablets, granules, pills, a syrup, or a beverage.
